(19) United States
(12) Patent Application Publication
(10) Pub. No.: US 2006/0148709 A1
(43) Pub. Date: Jul. 6, 2006

(54) Neuroprotective properties of GDF-15, a novel member of the TGF-Beta superfamily

(75) Inventors: Klaus Unsicker, Heidelberg (DE); Kerstin Krieglstein, Heidelberg (DE)

Correspondence Address: WEINGARTEN, SCHURGIN, GAGNEBIN & LEBOVICI LLP, TEN POST OFFICE SQUARE, BOSTON, MA, 02109, US

(73) Assignee: BIOPHARM GESELLSCHAFT ZUR BIOTECHNOLOGISCHEN ENTWICKLUNG VON PHARMAKA mbH

(21) Appl. No.: 11/372,281

(22) Filed: Mar. 9, 2006

Related U.S. Application Data
(63) Continuation of application No. 10/009,431, filed on Feb. 13, 2002, now abandoned.

(30) Foreign Application Priority Data
May 17, 1999 (EP) 99 109 714.8
Jul. 29, 1999 (EP) 99 114 853.7

(51) Int. Cl. A61K 48/00; A61K 38/18

(52) U.S. Cl. 514/12; 514/44

(57) ABSTRACT

The present invention relates to a transforming growth factor-beta (TGF-β)-like protein which is derived from neurons and glial cells, and which has a neurotrophic effect on dopaminergic (DAergic) neurons, to nucleic acids coding for the protein, to a vector containing the nucleic acids, to host organisms containing the nucleic acids or the vector, to antibodies directed against the protein, to methods for the production of the nucleic acids, the vector or the protein, to a pharmaceutical composition for the treatment of neurodegenerative disorders in mammals and to a diagnostic kit for the detection of said disorders.

This application is a continuation of U.S. patent application Ser. No. 10/009,431 filed Feb. 13, 2002, entitled NEUROPROTECTIVE PROPERTIES OF GDF-15, A NOVEL MEMBER OF THE TGF-BETA SUPERFAMILY; which was a §371 national stage of International Application PCT/EP00/04445 filed May 16, 2000; which claims benefit of European application No. 99109714.8 filed May 17, 1999 and European application No. 99114853.7 filed Jul. 29, 1999.

The present invention relates to a transforming growth factor-beta (TGF-β)-like protein which is derived from neurons and glial cells, and which has a neurotrophic effect on dopaminergic (DAergic) neurons, to nucleic acids coding for the protein, to a vector containing the nucleic acids, to host organisms containing the nucleic acids or the vector, to antibodies directed against the protein, to methods for the production of the nucleic acids, the vector or the protein, to a pharmaceutical composition for the treatment of neurodegenerative disorders in mammals and to a diagnostic kit for the detection of said disorders.

Members of the TGF-β superfamily are known for their important multifunctional implications in development and maintenance, such as the organization of the body plan, regulation of cell proliferation, differentiation, and cell survival. The still expanding TGF-β superfamily includes the TGF-β isoforms 1 to 5, activins, inhibins, bone morphogenetic proteins (BMPs), growth/differentiation factors (GDFs), mullerian-inhibiting substance, Drosophila decapentaplegic gene complex, Xenopus Vg-1 gene, and a growing subfamily of glial cell line-derived growth factors (GDNFs) and related proteins. All members of the TGF-β superfamily share several homologous structures. They are synthesized as large precursor molecules containing a biologically inactive pro-domain which can be secreted as a complex with the mature carboxyterminal portion. Furthermore, the mature bioactive proteins are generated by proteolysis using a characteristic cleavage site. Most notably, the mature carboxy-terminal segments contain a highly conserved cystein knot.

As TGF-β-like proteins are also implicated in the regulation of neuronal stem cell proliferation and maintenance of neurons there is a great demand for novel members of this protein family.

Accordingly, the technical problem underlying the present invention is to provide novel compounds relating to TGF-β-like proteins having neurotrophic activities which are suitable for the treatment and diagnosis of neurodegenerative disorders.

The solution to the above technical problem is achieved by providing the embodiments as characterized in the claims.

In particular, the present invention relates to a nucleic acid containing a nucleotide sequence encoding the primary amino acid sequence of a TGF-β-like protein or a functionally active derivative or part thereof which is derived from neurons and glial cells and which has a neurotrophic effect on DAergic neurons.

The terms “nucleic acid” and “nucleotide sequence” refer to endogenously expressed, semi-synthetic, synthetic or chemically modified nucleic acid molecules, preferably consisting substantially of deoxyribonucleotides and/or ribonucleotides and/or modified nucleotides. Further, the term “nucleotide sequence” may comprise exons, wherein the nucleotide sequence encodes the primary amino acid sequence and may be degenerated based on the genetic code. The term “primary amino acid sequence” refers to the sequence of amino acids irrespective of tertiary and quaternary protein structure.

The term “TGF-β-like protein” refers to proteins displaying the characteristics of the TGF-β superfamily, especially a conserved cystein rich motif, and comprises both the large precursor molecules containing a pro-domain as well as the mature bioactive proteins which are generated by proteolysis using a characteristic cleavage site.

The terms “functionally active derivative” and “functionally active part” refer to a proteinaceous compound exhibiting at least a neurotrophic effect on DAergic neurons. The functionally active form of the above-defined TGF-β-like protein may be a monomeric, dimeric and/or oligomeric form, as well as a heterooli-gomeric form, e.g. a heterodimer, comprising at least two different monomers of TGF-β-like proteins having neurotrophic activity.

The expression “derived from neurons and glial cells” means that the gene coding for the protein is transcribed and/or translated in neurons and glial cells such as Purkinje cells and astrocytes such that the mRNA and/or the protein is detectable by methods known in the art such as in situ hybridization, RT-PCR, Northern or Western blotting.

The expression “neurotrophic effect on DAergic neurons” refers to a proteinaceous activity that may confer, by itself or in combination with other factors, survival and differentiation upon DAergic neurons within the nanomolar range or below.

In a preferred embodiment of the above defined nucleic acid the neurons and glial cells are of mammalian origin, e.g. human, mouse or rat.

In a further preferred embodiment, the TGF-β-like protein protects against neurodegenerative events. Such neurodegenerative events may be e.g. mediated by oxidative damage, free radicals, mediators or executors of neuronal death programs such as caspases, pro- and anti-apoptotic members of the bcl-2 family. A toxic radical damage may be mediated by iron, e.g. Fe-ions, NO and other radical donors. Therefore, the nucleic acid as defined above encodes a TGF-β-like protein which is able to protect DAergic neurons against intoxication by iron, which is suggested to cause Parkinson's disease (PD).

In a further preferred embodiment the nucleic acid according to the present invention comprises at least the nucleotide sequence shown in FIG. 7A (SEQ ID NO. 1) or the nucleotide sequence shown in FIG. 8A (SEQ ID NO. 2) or nucleotides 40 to 333 of the nucleotide sequence shown in FIG. 8A (SEQ ID NO. 2) or mutants of such nucleic acids leading to the expression of functionally active polypeptides. Examples of such mutations include deletions, insertions and substitutions of one or more nucleotides such as mutations which lead to conservative amino acid substitutions, e.g., such mutations in the range of nucleotides 40 to 333 of the nucleotide sequence shown in FIG. 8A (SEQ ID NO. 2), i.e., the region of the nucleotide sequence encoding the 7 Cys-knot region, which is highly conserved in TGF-β-like proteins.

A further subject of the present invention relates to a vector containing at least the nucleic acid as defined above. The term “vector” refers to a DNA and/or RNA replicon that can be used for the amplification and/or expression of the above defined nucleotide sequence. The vector may contain any useful control units such as promoters, enhancers, or other stretches of sequence within the 5′ regions of the sequence serving for the control of its expression. The vector may additionally contain sequences within the 5′ and/or 3′ region of the nucleotide sequence, that encode amino acid sequences such as a His-tag which are useful for the detection and/or isolation of the protein encoded by the nucleotide sequence. Furthermore, the vector may contain sequence elements within the 5′ and/or 3′ region of the nucleotide sequence encoding amino acid sequences which serve for the targeting of the protein encoded by the nucleotide sequence to nerve tissues and/or for the penetration of the blood/brain barrier. Examples of suitable vectors are baculovirus vectors.

Another embodiment of the present invention relates to a host organism containing the nucleic acid or the vector, as defined above. The term “host organism” comprises a virus, a bacterium such as Escherichia coli, a fungus, a plant, a mammal or an insect or parts such as cells, e.g. Sf9 cells, thereof.

A further embodiment of the present invention relates to the protein itself, which is encoded by the nucleic acid as defined above. Examples of the primary amino acid sequence of the protein according to the present invention are given in FIGS. 7B (SEQ ID NO. 3) and 8B (SEQ ID NO. 4), respectively. Further examples of the primary amino acid sequence of the protein according to the present invention comprise amino acid residues 14 to 111 of the sequence shown in FIG. 8B (SEQ ID NO. 4) as well as homologs thereof having conservative amino acid substitutions.

A further subject of the present invention relates to an antibody, which may be monoclonal or polyclonal, or a functional fragment thereof directed against the protein or a functional derivative or part thereof as defined above. Further subjects of the present invention relate to an antagonist directed to the above-defined protein and to an agonist as a substitute for the above-defined protein.

A modulation of the functional activity of the above-defined protein may also be achieved by altering the expression of the nucleotide sequence of the above-defined nucleic acid as compared to the expression level in a normal cell. For example, an antisense nucleic acid masking the mRNA or a ribozyme cleaving the mRNA may be used to inhibit the expression. Alternatively, the efficiency of the promoter which regulates the expression of the nucleotide sequence of the above-defined nucleic acid may be influenced.

A further embodiment of the present invention relates to a method for the production of the nucleic acid, the vector, or the protein as defined above, comprising the steps of:
(a) cultivating the above-defined host organism in a suitable medium under suitable conditions; and
(b) isolating the desired product from the medium and/or the host organisms.

A preferred embodiment of the method for the production of the protein according to the present invention uses bacteria such as E. coli as the host organsim. The expression of the above-defined protein may then lead to a functionally inactive form, e.g. of amorphous aggregates within the bacterium known in the art as “inclusion bodies”. Therefore, the method of the present invention may further comprise steps serving for the refolding and/or modification of the isolated protein into a functionally active form which may be a monomeric, dimeric or oligomeric form. In particular, the present invention further comprises a method for the production of the biologically active dimeric form of the protein as defined above, preferably GDF-15, from its denatured or otherwise non-native form. This object of the present invention is achieved by the unexpected finding that considerable amounts of the desired dimeric products are obtained by subjecting the monomeric form of the protein according to the present invention to refolding conditions. Thus, the present invention also relates to dimeric biologically active GDF-15 which has been produced by the above-defined method.

A further embodiment of the present invention relates to a pharmaceutical composition comprising the nucleic acid or the vector or the protein or the antibody or the antagonist or the agonist as defined above, optionally in combination with a pharmaceutically acceptable carrier and/or diluent. The pharmaceutical composition may be used for the prevention and/or treatment of neurodegenerative disorders in mammals, preferably in humans. Furthermore, therapeutic techniques for the treatment of disorders which are associated with the expression of the nucleotide sequence of the nucleic acid according to the present invention may be designed using the above-mentioned agents which are capable of regulating the expression of the nucleotide sequence of the above-defined nucleic acid, e.g. antisense nucleic acids, ribozymes and/or agents for influencing promoter activity. The neurodegenerative disorders are preferably acute and/or chronic neurological and psychological disorders, and may be caused by stroke, parkinson's disease, Alzheimer's disease or other dementias, infections of the CNS and psychiatric disorders associated with disturbances in CNS transmitter systems such as depression and schizophrenia.

In a further preferred embodiment, the pharmaceutical composition acccording to the present invention further comprises, in addition to the nucleic acid or the vector or the protein or the antibody or the antagonist or the agonist as defined above, one or more other agents having neurotrophic activity. Preferred agents are, e.g., cytokines or functionally active derivatives or parts thereof. Preferred cytokines used in the pharmaceutical composition according to the present invention may be selected from the group consisting of GDF such as GDF-5, GDF-6, GDF-7, GDF-8 and GDF-9, GDNF, TGF such as TGF-α or TGF-β, e.g. TGF-β1, TGF-β2 or TGF-β3, activin A, BMP such as BMP-2, BMP-4, BMP-6, or BMP-7, BMP-11, BMP-12, BDNF, NGF, neurotrophines such as NT-3 or NT-4, EGF, CNTF and FGF such as FGF-2. The term “GDNF” includes GDNF, neurturin and persephin.

A further subject of the present invention relates to a diagnostic kit comprising the nucleic acid, the vector, the protein and/or the antibody as defined above, for the detection of neurodegenerative disorders and/or infections of the CNS such as meningitis, e.g. a bacterial meningitis, in mammals, preferably humans. Examples of other neurodegenerative disorders are as defined above.

### BRIEF DESCRIPTION OF THE DRAWINGS

Other features and advantages of the invention will be apparent from the following description of the preferred embodiments thereof and from the claims, taken in conjunction with the accompanying drawings, which show:

FIGS. 1A-1D Localization of GDF-15 in the CNS. (A) Photographic image of an in situ hybridization of an adult rat choroid plexus performed with rat specific GDF-15 antisense-RNA probes. (B) Photographic image of an immunoblot analysis of human cerebrospinal fluid (CSF) under reducing conditions with purified GDF-15 antiserum. (C) RT-PCR of different P0 rat brain regions (pons, medulla oblongata, cortex, hippocampus, striatum), dorsal root ganglia (DRG), cultured primary astrocytes (astr.), oligodendroglial cell line OLI-neu (OLI), and cultured oligodendroglial progenitors (O-2A). (D) Immunoblot analyis under native conditions of the corresponding brain areas and cells of (c) with purified GDF-15 antiserum.

FIG. 2 Image of Western blot analysis of GDF-15 in human CSF under reducing conditions. Molecular weight marker (St.). CSF sample of a patient with bacterial meningitis (lane 1). CSF sample of a control patient (lane 2).

FIG. 3 Graphic representation of experiments showing the survival effect of GDF-15 in mesencephalic neuron cultures. Numbers of surviving tyrosine hydroxylase (TH)-immunoreactive neurons of mesencephalic cultures (E15/DIV7) treated with medium only (control), purified lysate from uninfected Sf9 cells (baculo control), GDF-15 (0.01 to 1 ng/ml) purified from infected Sf9 cell lysate, and GDNF (10 ng/ml). Data are given as mean±SEM (n=3), P-values derived from Student's t-test are ***P<0.001, **P<0.01 for increased survival as compared with control cultures.

FIGS. 4A-4B Protective effect of GDF-15 in Fe²⁺ (100 μM) treated cultures. (A) Graphic representation of numbers of surviving TH-immunoreactive neurons of mesencephalic cultures (E15/DIV7) treated with or without Fe²⁺ in medium only (control), in presence of NT-4 (10 ng/ml), and in presence of GDF-15 (10 ng/ml). (B) Graphic representation of the percentage of surviving TH-immunoreactive neurons of mesencephalic cultures (E15/DIV7) treated with Fe²⁺ in medium only (control), in presence of NT-4 (10 ng/ml), and in presence of GDF-15 (10 ng/ml). Values of cultures without addition of iron are set to 100%. Data are given as mean±SEM (n=3), P-values derived from Student's t-test are *P<0.05 for increased survival as compared with control cultures.

FIGS. 5A-5B In vivo neurotrophic effects of GDF-15. (A) Graphic representation of amphetamine rotation data of rats with unilateral 6-OHDA (6-hydroxydopamine) lesions. Rotations per minute were monitored for 60 min beginning 5 min after amphetamine (5 mg/kg i.p.) administration. (B) Graphic representation of counts of TH-positive neurons in SNpc. Values are given as percentage of TH-positive neurons of the lesioned as compared to the unlesioned side. Data are given as mean+/−SEM (n=4). P-values derived from Student's t-test are *P<0.05, **P<0.01, ***P<0.001.

FIGS. 6A-6B Signalling of GDF-15 through Smad proteins. (A) Graphic representation of experiments demonstrating the activation of the Smad Binding Element (SBE) by TGF-β1 and GDF-15 in transient transfected hFob cells. (B) Graphic representation of experiments showing that the PAI-1 promoter which is exclusively activated by TGF-β1 to -β3 in stable transfected MLEC cells does not respond to GDF-15.

FIGS. 7A-7B (A) cDNA (SEQ ID NO. 1) and (B) corresponding amino acid sequence of human pre-pro-mature GDF-15 (SEQ ID NO. 3). Nucleotides and amino acids are abbreviated according to the international one letter codes.

FIGS. 8A-8B (A) cDNA (SEQ ID NO. 2) and (B) corresponding amino acid sequence of human mature GDF-15 (SEQ ID NO. 4).

The following non-limiting example illustrates the invention:

### EXAMPLE

Identification of GDF-15

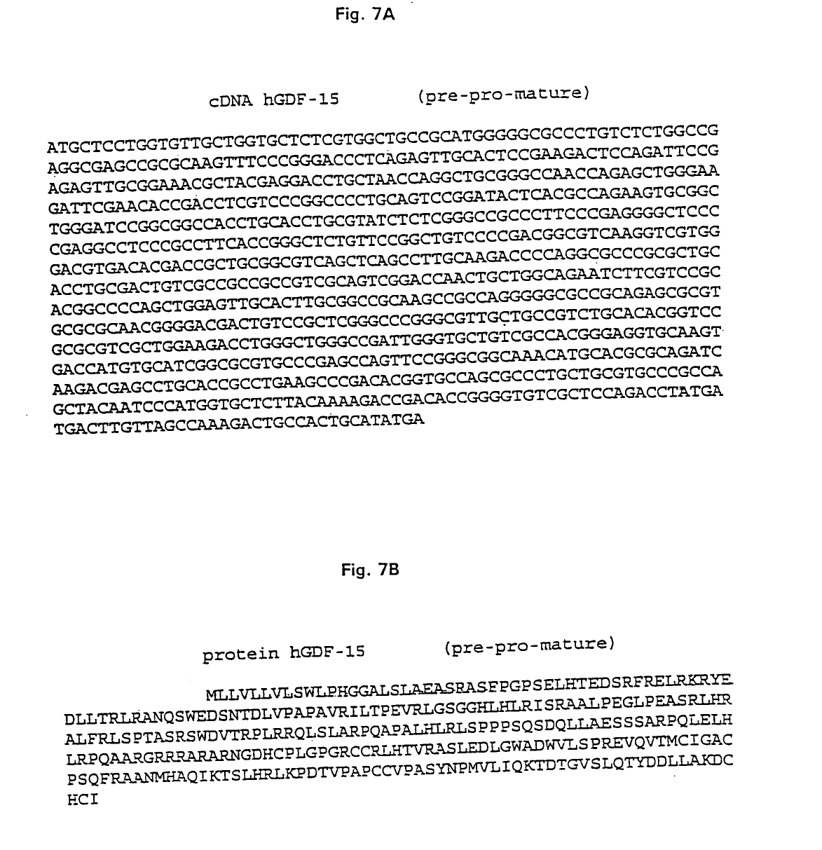

Using the conserved cystein knot motif of TGF-β-like proteins, a combined approach employing RT-PCR and library screening revealed the full-length cDNA sequence of a novel member of the TGF-β superfamily derived from neurons. The cDNA has the sequence shown in FIG. 7A corresponding to the amino acid sequence shown in FIG. 7B.

According to a possible alternative translation start codon which is located 39 nucleotides upstream from the first nucleotide of the sequence shown in FIG. 7A (SEQ ID NO. 1), the corresponding protein may also comprise 13 additional amino acids (MPGQELRTLNGSQ) (SEQ ID NO. 5) N-terminal to the sequence shown in FIG. 7B (SEQ ID NO. 3).

The protein, which is named GDF-15, was recombinantly expressed using the baculovirus system. Furthermore, an antibody against a specific peptide derived from the murine and rat C-terminal sequence (HRTDSGVSLQTYDDL) (SEQ ID NO. 6) has been developed. Due to the high homology of the corresponding region of the human sequence (QKTDTGVSLQTYDDL) (SEQ ID NO. 7), this antibody recognizes also human GDF-15.

Localization of GDF-15 in the CNS

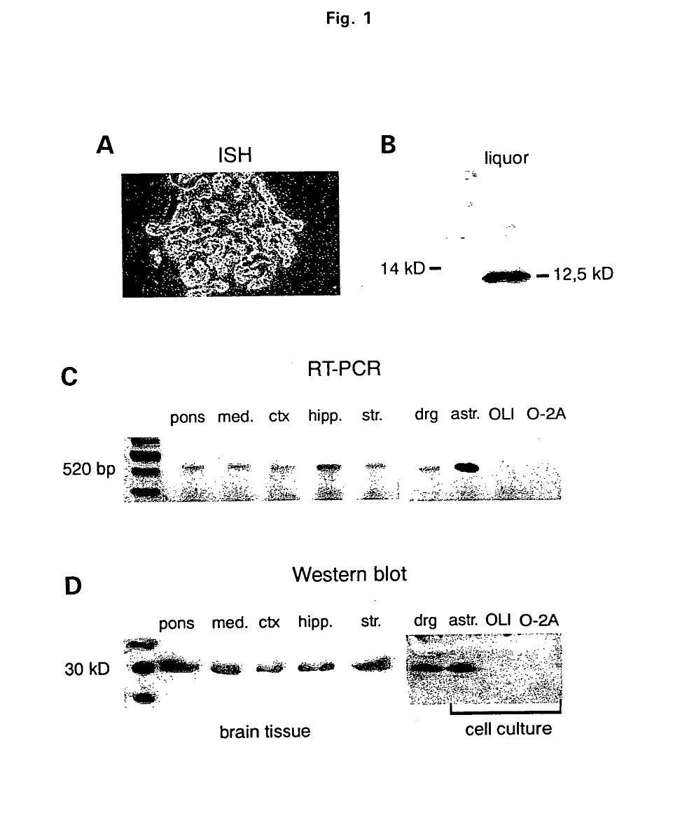

In situ hybridization with GDF-15 antisense RNA probes, RT-PCR as well as Western blot analyses were performed to study the distribution of GDF-15 in the CNS (FIG. 1). In situ hybridization revealed signals in neurons, especially Purkinje cells, in the cerebellum, and strong expression in the choroid plexus (FIG. 1A) of newborn and adult rats. RT-PCR and Western blotting of samples taken from different regions of newborn and adult rat brains and peripheral nervous system extended these results by detecting mRNA and protein in pons, medulla oblongata, midbrain, striatum, hippocampus, cortex, and dorsal root ganglia (FIGS. 1C, D). Highest levels of mRNA expression were found in the choroid plexus (FIG. 1A). Antibodies raised against the above C-terminal peptide were used for Western blots. Analysis of samples of different brain areas of newborn rats revealed one distinct band at 31 kDa (FIG. 1D). The relative mass of cellular GDF-15 is in good agreement with the theoretical molecular weight of 31 kDa of the pro-protein.

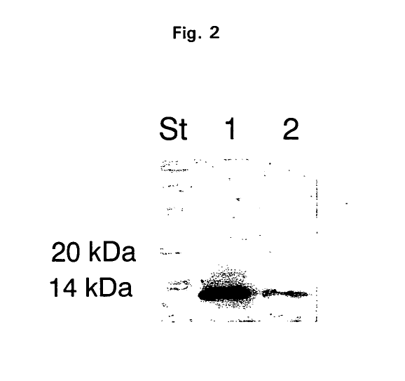

As GDF-15 is abundant in the choroid plexus, the presence of the protein in CSF of healthy human subjects as well as patients with different neurological disorders was also tested. In contrast to the intracellular protein detected in brain samples, CSF samples revealed a single band at about 12 kDa under reducing conditions representing the secreted mature portion of GDF-15. Highest amounts of protein in CSF were seen in patients with bacterial meningitis (FIG. 2). Taken together these data provide evidence that GDF-15, a novel member of the TGF-β superfamily, is widely expressed in various regions of the CNS including CSF and peripheral nervous system. Furthermore, GDF-15 is significantly increased in the CSF of patients with inflammatory neurological disease providing the opportunity to employ antibodies to GDF-15 as diagnostic tools in neurological disease.

Production of Dimeric, Biologically Active GDF-15

2 μg of monomeric GDF-15 protein (e.g. produced in bacteria such as E. coli) is dissolved in 2917.7 μl solubilisation buffer (1 M NaCl, 50 mM Tris-HCl, 50 mM EDTA, pH 9.5). To the thus dissolved protein, the following is added (resulting in a total volume of 3580 μl):
35.8 μl 100 mM oxidized Glutathion (GSSG)
35.8 μl 200 mM reduced Glutathion (GSH)
590.7 μl CHAPS (3-[(Cholamidopropyl)-dimethylamino]-1-propane sulfonate)

After incubation at 20 to 22° C. for 48 h, more than 80%, typically 90%, of the monomeric protein is refolded into the desired dimeric product. The separation of the dimer is performed by standard chromatographic methods such as reverse phase HPLC.

Functional Studies Using Recombinant Human GDF-15

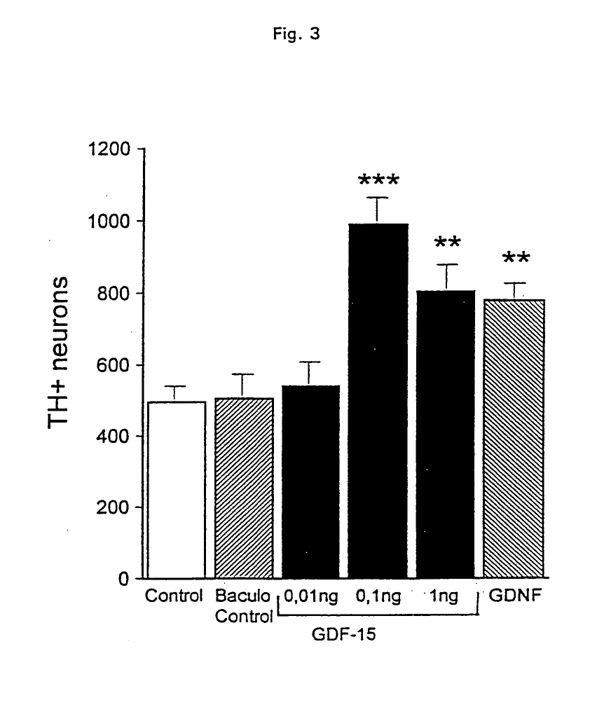

Using the baculovirus system, the mature part of the human recombinant GDF-15 protein was expressed in Sf9 cells. However, the same results in all functional studies are obtained when using recombinant human GDF-15 expressed in bacteria which has been renatured by the above-described refolding method. Western blot showed the monomeric or dimeric form of the recombinant protein under reducing and non-reducing conditions, respectively. Following purification, the protein was tested for its survival effects on rat embryonic midbrain DAergic neurons. Addition of recombinant GDF-15 to cultures of E14 midbrain cells augmented numbers of surviving tyrosine hydroxylase (TH)-positive neurons after 7 days in vitro compared to control cultures (FIG. 3). The dopaminotrophic effect of GDF-15 is comparable to the documented survival promoting activity of other members of the TGF-β superfamily and the neutrophin family (e.g. TGF-β, GDNF-subfamily members, or BDNF). Analysis of midbrain cultures using immunocytochemistry and antibodies to the astrocyte-specific intermediate filament protein GFAP and assays for cell proliferation provided evidence that GDF-15 application did not exert its survival promoting effect through numerically increasing cells and promoting maturation of astrocytes, a well-established source of neurotrophic factors. This provides evidence that GDF-15 affects dopaminergic neurons directly rather than indirectly, as shown for FGF-2 or BMPs.

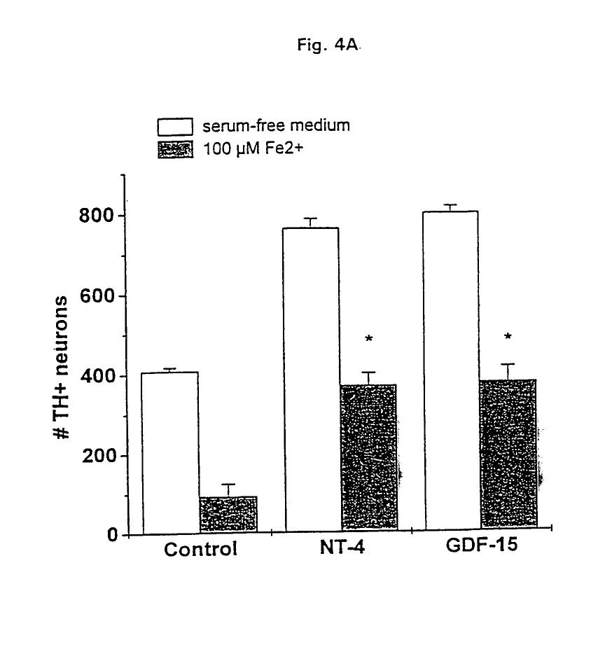

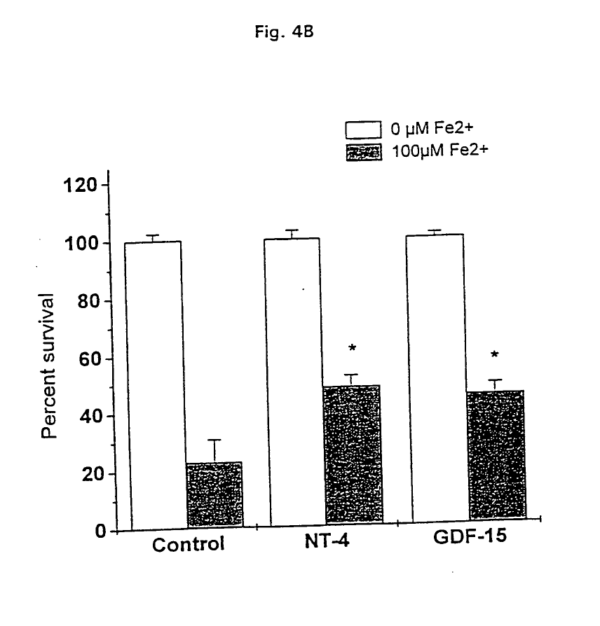

In order to investigate whether GDF-15 is also able to protect DAergic neurons against a likely cause of PD, i.e. iron intoxication, its effects on iron-intoxicated mesencephalic neurons was examined (FIGS. 4A, B). Exposure of cultures to iron (Fe²⁺) caused a 80% reduction in neuronal survival compared to untreated control cultures. Cell losses were reduced to 50% when cultures were co-treated with Fe²⁺ and GDF-15. These data strongly suggest that GDF-15 protects DAergic neurons against iron-mediated (oxidative) damage. The data also support the use of GDF-15 as an agent to prevent or slow down neurodegenerative events mediated by free radicals, oxidative stress, mediators and executors of neuronal death programs.

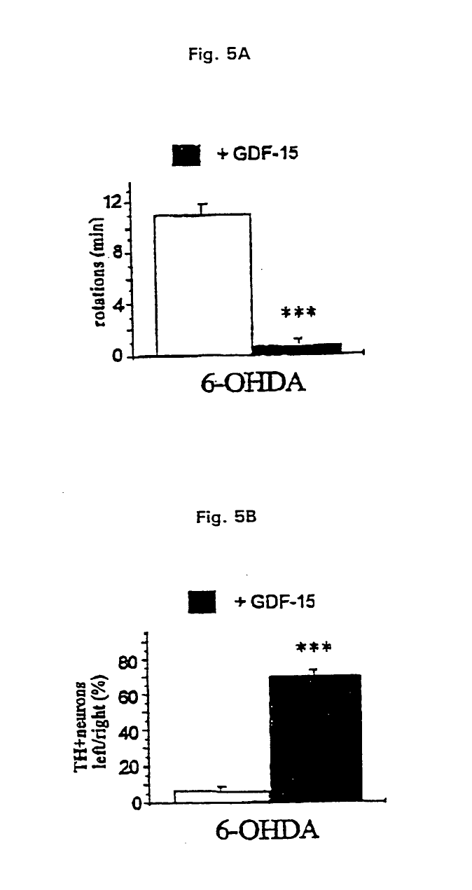

Furthermore, it was established that GDF-15 also protects lesioned DAergic midbrain neurons in vivo. The nigrostriatal system of adult rats was lesioned by an unilateral injection of 6-hydroxydopamine (6-OHDA) just above the left substantia nigra (SN). The results of these experiments are shown in Tables 1A and B, respectively. The data shown in Table 1B are also represented graphically in FIG. 5A.

TABLE 1
Amphetamine rotation data
A: Rotations per minute for 60 min beginning 5 min after
amphetamine administration (5 mg/kg i.p.)

| Rat no. | Treatment | Rotations per min |
|---|---|---|
| 1 | 6-OHDA | 13 |
| 2 | 6-OHDA | 11 |
| 3 | 6-OHDA | 9 |
| 4 | 6-OHDA | 11 |
| 5 | 6-OHDA + GDF-15 | 0 |
| 6 | 6-OHDA + GDF-15 | 1 |
| 7 | 6-OHDA + GDF-15 | 2 |
| 8 | 6-OHDA + GDF-15 | 0 |

B: Mean values

| Treatment | Rotations per min (mean ± SD) |
|---|---|
| 6-OHDA | 11.0 ± 1.4 |
| 6-OHDA + GDF-15 | 0.8 ± 0.8 |

All rats displayed the typical features of amphetamine challenge, such as stereotypy and piloerection. Rats which had been treated with 6-OHDA only showed ratation rates of 11.0±1.41 (mean±SD), indicating at least 95% depletion of the nigrostriatal pathway (Ungerstedt at al. (1970), Brain. Res., 24, 485-493). In contrast, rats which were also treated with GDF-15 rotated at very low rates (0.75±0.83), showing that this protein effectively prevented 6-OHDA-induced depletion of dopamine in the left striatum; cf. also FIG. 5A.

Furthermore, in order to confirm that the above prevention of 6-OHDA-induced dopamine depletion in the left striatum is due to a neuroprotective effect of GDF-15 on neurons in the SN, the SN pars compacta (SNpc) was analysed immunocytochemically. Counts of TH-positive neurons in the SNpc measured at three individual levels are shown in Table 2A and the mean values for each rat are given in Table 2B, respectively. The overall mean values for the 6-OHDA-treated (n=4) and for the rats which were co-treated with 6-OHDA and GDF-15 (n=4) are shown in Table 2C. The data shown in Table 2C are also represented graphically in FIG. 5B. The results show that the co-treatment of the rats with 6-OHDA and GDF-15 led to a 10-fold increase in the count of TH-positive neurons in the left striatum compared to the treatment with 6-OHDA alone. Therefore, GDF-15 prevents 6-OHDA-induced depletion of dopamine in the left striatum due to its strong neuroprotective effect on TH-immunoreactive neurons.

TABLE 2
TH-immunocytochemistry data
A: Counts of TH-positive neurones in substantia nigra pars
compacta at three levels; −2.8, −3.0 and −3.2, relative to
bregma (according to Pellegrino et al., A stereotaxic atlas of
the rat brain. Plenum Press, New York, 1979)

| | | TH counts | | | TH counts | | | TH counts | | |
|---|---|---|---|---|---|---|---|---|---|---|
| | | (−2.8) | | | (−3.0) | | | (−3.2) | | |
| Rat | | | | L/R | | | L/R | | | L/R |
| no. | Treatment | Right | Left | (%) | Right | Left | (%) | Right | Left | (%) |
| 1 | 6-OHDA | 102 | 6 | 5.9 | 121 | 8 | 6.6 | 125 | 11 | 8.8 |
| 2 | 6-OHDA | 114 | 11 | 9.6 | 117 | 10 | 8.5 | 114 | 12 | 10.5 |
| 3 | 6-OHDA | 98 | 3 | 3.1 | 104 | 4 | 3.8 | 106 | 9 | 8.5 |
| 4 | 6-OHDA | 99 | 3 | 3.0 | 112 | 7 | 6.3 | 97 | 6 | 6.2 |
| 5 | 6-OHDA + GDF- | 107 | 69 | 64.5 | 111 | 73 | 65.8 | 114 | 81 | 71.1 |
| | 15 | | | | | | | | | |
| 6 | 6-OHDA + GDF- | 110 | 71 | 64.5 | 109 | 65 | 59.6 | 120 | 84 | 70.0 |
| | 15 | | | | | | | | | |
| 7 | 6-OHDA + GDF- | 114 | 78 | 68.4 | 123 | 101 | 82.1 | 126 | 97 | 77.0 |
| | 15 | | | | | | | | | |
| 8 | 6-OHDA + GDF- | 115 | 80 | 69.6 | 118 | 95 | 80.5 | 118 | 89 | 75.4 |
| | 15 | | | | | | | | | |

B: Mean values of individual rats

| | | TH counts (mean ± SD) | | |
|---|---|---|---|---|
| Rat no. | Treatment | Right | Left | L/R (%) |
| 1 | 6-OHDA | 116.0 ± 10.0 | 8.3 ± 2.1 | 7.1 ± 1.2 |
| 2 | 6-OHDA | 115.0 ± 1.4 | 11.0 ± 0.8 | 9.5 ± 0.8 |
| 3 | 6-OHDA | 102.7 ± 3.4 | 5.3 ± 2.6 | 5.1 ± 2.4 |
| 4 | 6-OHDA | 102.7 ± 6.6 | 5.3 ± 1.7 | 5.2 ± 1.5 |
| 5 | 6-OHDA + GDF-15 | 110.7 ± 2.9 | 74.3 ± 5.0 | 67.1 ± 2.9 |
| 6 | 6-OHDA + GDF-15 | 113.0 ± 5.0 | 73.3 ± 7.9 | 64.7 ± 4.2 |
| 7 | 6-OHDA + GDF-15 | 121.0 ± 5.1 | 92.0 ± 10.0 | 75.8 ± 5.7 |
| 8 | 6-OHDA + GDF-15 | 117.0 ± 1.4 | 88.0 ± 6.2 | 75.2 ± 4.5 |

C: Mean values of 6-OHDA-treated rats and mean values after co-
treatment with 6-OHDA plus GDF-15

| | TH counts (mean ± SD) | | |
|---|---|---|---|
| Treatment | Right | Left | Left/Right (%) |
| 6-OHDA | 109.1 ± 6.4 | 7.5 ± 2.4 | 6.7 ± 1.8 |
| 6-OHDA + GDF-15 | 115.4 ± 3.9 | 81.9 ± 8.2 | 70.7 ± 4.9 |

In summary, the above in vivo studies demonstrate that injections of GDF-15 immediately prior to 6-OHDA above the left SN and into the left lateral ventricle prevented 6-OHDA-induced pathological rotation behavior (FIG. 5A) and significantly reduced losses of DAergic SN neurons (FIG. 5B). Together, these data show that GDF-15 can be profitably employed to ameliorate consequences of nigrostriatal degeneration in Parkinson's disease.

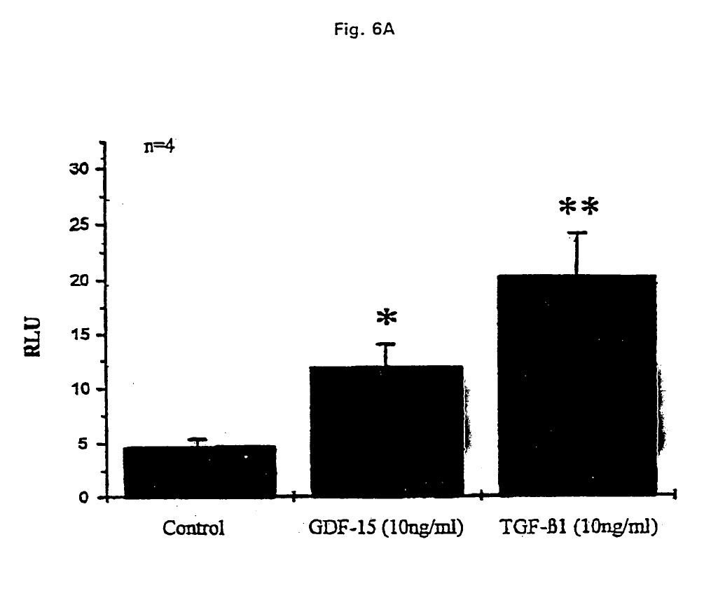

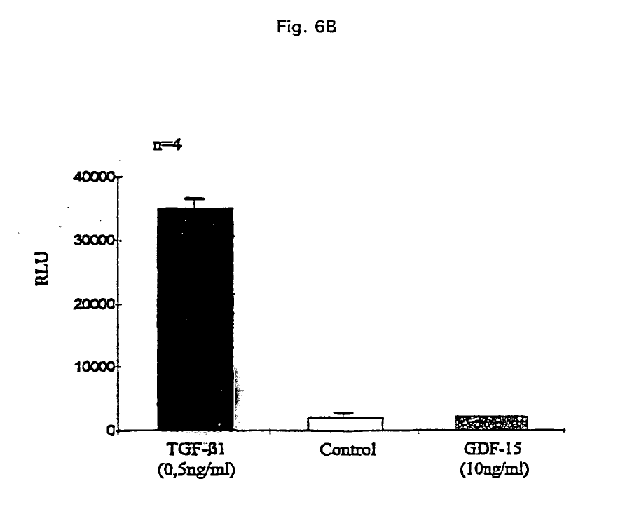

Using the plasmid Smad Binding Element (pSBE) which is activated by TGF-β1, OP-1 (also referred to as BMP-7), activin, BMP-2 and GDF-5, the further question was addressed as to whether GDF-15 is able to induce intracellular signal transduction through Smad proteins. Transient transfection of the human osteoblast cell line (hFob) with SBE showed that GDF-15 administration increased the luciferase signal (FIG. 6A). These results demonstrate that GDF-15 activates the Smad responsive promoter element of the reporter gene construct. In a further experiment the inducibility of the Plasminogene Activator Inhibitor promoter (PAI) in stable transfected Mink Lung Epithelial Cells (MLEC) by GDF-15 was tested. The MLEC assay, which is exclusively sensitive for TGF-β1, -β2, and -β3, revealed no effect of GDF-15 (FIG. 6B). Since Smad2 and Smad3 phosphorylation is specifically associated with the TGF-β-mediated activation of TGF-β receptors, it is concluded that GDF-15 seems not to signal through the Smad2/3 pathway. With regard to the GDF-15-dependent activation of SBE, which is a response element for both, the Smad2/3, and the BMP-mediated Smad1/5 pathway, it appears that GDF-15 exerts its cellular effects by binding to BMP-like receptors.

### SUMMARY

In conclusion, a novel neurotrophic molecule derived from neuron cells belonging to the TGF-β superfamily, GDF-15, was discovered, cloned, expressed and functionally characterized.

In the nervous system, GDF-15 mRNA and protein can be detected, e.g. in midbrain, striatum and in cortex, but highest levels of the mRNA and the protein are found in the choroid plexus and spinal fluid (CSF), respectively. Interestingly, levels of protein in CSF are increased in certain neurological disorders, e.g. in patients with bacterial meningitis. In order to elucidate its functions, the mature form of human GDF-15 was recombinantly expressed using a baculovirus expression system. Expression resulted in the synthesis of the biologically active dimeric form of the protein. In vitro experiments using dissociated cell cultures of embryonic rat midbrain neurons revealed that GDF-15 can act as a neurotrophic factor for DAergic midbrain neurons which degenerate in Parkinson's disease (PD). GDF-15 is also able to protect these neurons against intoxication by iron, which may be causal to PD. Furthermore, it could be demonstrated that GDf-15 also exhibits its neuroprotective effect in vivo. Concerning the signalling pathway GDF-15 acts upon, it was established that GDF-15 is able to induce intracellular signal transduction through Smad proteins.

Therefore, it can be concluded that GDF-15 has important functions in the developing, mature, and lesioned brain involving options to use GDF-15 for the treatment and diagnosis of acute and chronic neurological and psychological disorders, such as stroke, Alzheimer's disease and other demetias, and psychiatric disorders associated with disturbances in CNS transmitter systems.

Methods for in Vivo Studies Demonstrating the Protective Effect of GDF-15 on 6-OHDA-Lesioned Nigrostriatal Neurons

Adult female Wistar rats were anaesthetised using ketamine (75 mg/kg i.p.) and xylazinum (15 mg/kg i.p.) and placed in a Kopf stereotaxic frame. GDF-15 was used at a final concentration of 2 μg/μl in 10 mM phosphate-buffered saline (PBS), pH 7.4. Four rats received injections of 20 μg GDF-15 just above the left substantia nigra (SN) and 20 μg GDF-15 into the left lateral ventricle (LV). This was followed immediately by an injection of 6-hydroxydopamine hydrobromide (8 μg as the free base in 4 μl 0.9% saline with 0.1% ascorbic acid) into the left medial forebrain bundle (MFB). Four additional rats received 6-OHDA only. Stereotaxic co-ordinates (Pellegrino et al. A stereotaxic atlas of the rat brain. Plenum Press, New York, 1979) were as follows: AP −3.0, LV +2.5, DV −8.5 for the SN; AP +1.0, LV +1.2, DV −3.5 for the LV; AP −2.2, LV +1.5, DV −7.9 for the MFB. All rats were tested behaviourally at seven days after surgery. Ipsilateral rotations were counted over a 60 min period beginning 5 min after (+)-ampthetamine sulphate administration (5 mg/kg, i.p.). At ten days after surgery, all rats were terminally anaesthetised with chloroform/ether and perfused intracardially with 200 ml of cold 0.1 M phosphate-buffered saline (PBS), pH 7.4, containing 500 Units heparin, followed by 300 ml freshly prepared 4% paraformaldhyde in PBS. Brains were removed and placed in 4% paraformaldehyde in PBS overnight, cryoprotected in 30% sucrose in PBS and then frozen. Serial 30 μm coronal cryosections through the SN pars compacta (SNpc) were cut and stained immunocytochemically for tyrosine hydroxylase (TH). Sections were incubated in blocking solution (3% normal goat serum, 0.2% Triton X-100 in PBS) overnight at 4° C., then in a 1:2000 solution of rabbit antiserum to TH (Affiniti Labs, U.K.) in blocking solution overnight at 4° C. Sections were washed five times in PBS containing 0.02% Triton X-100, then incubated in a solution of 1:1000 horse radish peroxidase-linked anti-rabbit IgG (Vector Labs) overnight at 4° C. After washing as before, TH immunostaining was visualised using 3,3′-diaminobenzidine as the chromogen. Sections were mounted onto gelatinised slides, dehydrated in alcohol, cleared in xylene and mounted in DePeX® (Bioproducts, Heidelberg, Germany). TH-immunoreactive neurons were counted in the SNpc on both sides of the brain at each of three levels; −2.8, −3.0, −3.2, with respect to bregma (Pellegrino et al., 1979).

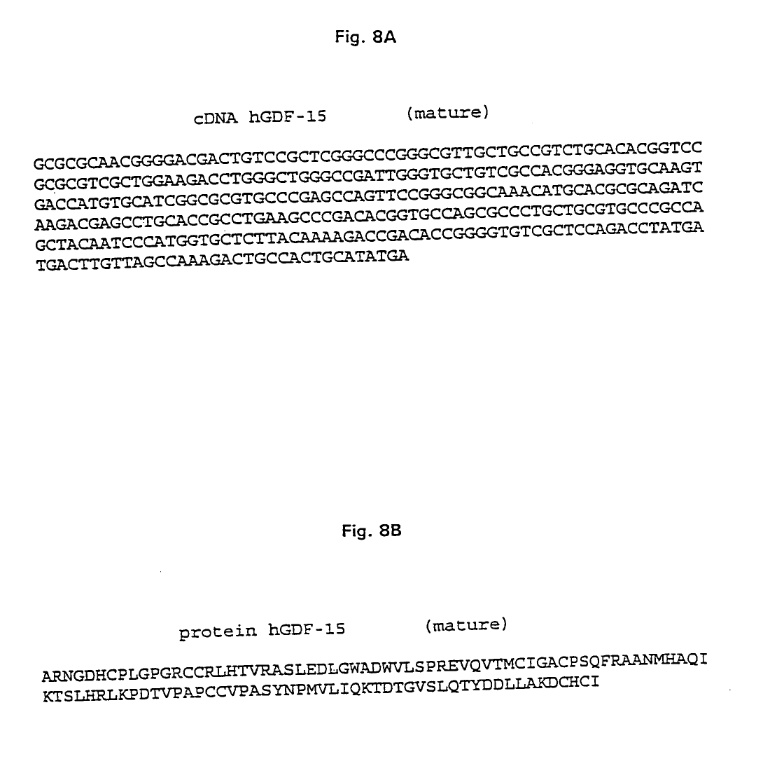

1. A method for protecting dopaminergic neurons in a patient against neuronal degeneration, said method comprising the steps of:
providing a patient in need of said protection; and
administering to said patient at least one substance selected from the group consisting of:
a) a GDF-15 protein of the TGF-β superfamily or a functionally active derivative or part thereof;
b) a protein comprising the sequence shown in FIG. 7B (SEQ ID NO. 3), or homologs thereof having conservative amino acid substitutions;
c) a protein comprising the sequence shown in FIG. 8B (SEQ ID NO. 4), or homologs thereof having conservative amino acid substitutions;
d) a protein comprising amino acids 14 to 111 of the sequence shown in FIG. 8B (SEQ ID NO. 4), or homologs thereof having conservative amino acid substitutions;
e) a nucleotide sequence encoding a protein according to a) to d);
f) a vector containing at least the nucleotide sequence according to e); and
g) an agonist as a substitute of the protein according to a) to d).

2. The method according to claim 1, wherein said neuronal degeneration of dopaminergic neurons is caused by oxidation or free radical damage, or mediators or executors of neuronal death programs.

3. The method according to claim 2, wherein the mediators of free radical damage are selected from the group consisting of iron, nitrous oxide, and other free radical donors, and the mediators and executors of neuronal death programs are selected from the group consisting of caspases and pro- and anti-apoptotic members of the bcl-2 family.

4. The method according to claim 1, wherein said patient is a mammal suffering from a disorder characterized by a degeneration of dopaminergic neurons.

5. The method according to claim 4, wherein the disorders characterized by a degeneration of dopaminergic neurons are selected from the group consisting of acute and chronic neurological disorders.

6. The method according to claim 4, wherein the disorders characterized by a degeneration of dopaminergic neurons are selected from the group consisting of stroke, Parkinson's disease, Alzheimer disease, dementias, and infections of the central nervous system.

7. The method according to claim 1, further comprising administering to said patient one or more agents having neurotrophic activity or functionally active derivatives or parts thereof.

8. The method according to claim 7, wherein said one or more agents is selected from the group consisting of GDF, GDNF, TGF, activins, BMP, BDNF, NGF, EGF, CNTF and FGF.


SEQUENCE LISTING (7 sequences)

SEQ ID NO: 1 - DNA, 888 bp, Homo sapiens
atgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggacgactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcttacaaaagaccgacaccggggtgtcgctccagacctatgatgacttgttagccaaagactgccactgcatatga

SEQ ID NO: 2 - DNA, 339 bp, Homo sapiens
gcgcgcaacggggacgactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcttacaaaagaccgacaccggggtgtcgctccagacctatgatgacttgttagccaaagactgccactgcatatga

SEQ ID NO: 3 - protein, 295 aa, Homo sapiens [sequence omitted]

SEQ ID NO: 4 - protein, 112 aa, Homo sapiens
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 5 - protein, 13 aa, Homo sapiens
MPGQELRTLNGSQ

SEQ ID NO: 6 - protein, 15 aa, Artificial Sequence
  Description of Artificial Sequence Peptide derived from the murine and rat C-terminal sequence of GDF-15
HRTDSGVSLQTYDDL

SEQ ID NO: 7 - protein, 15 aa, Homo sapiens
  PEPTIDE (1)..(15): Peptide corresponds to amino acids 273 to 287 of human pre-pro-mature GDF-15
QKTDTGVSLQTYDDL